(19) United States
(12) Patent Application Publication
(10) Pub. No.: US 2008/0200568 A1
(43) Pub. Date: Aug. 21, 2008

(54) Genes Associated With Type ll Diabetes Mellitus

(76) Inventor: Stephanie Chissoe, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/063,043

(22) PCT Filed: Aug. 30, 2006
(86) PCT No.: PCT/US2006/033564

§ 371 Date: Feb. 6, 2008

Related U.S. Application Data
(60) Provisional application No. 60/712,655, filed on Aug. 30, 2005.

(51) Int. Cl. A61K 48/00; C12Q 1/68

(52) U.S. Cl. 514/789; 435/6

(57) ABSTRACT

Genes associated with Type II Diabetes Mellitus (T2DM) are identified and screening methods to identify chemical compounds that act on those targets for the treatment of T2DM or its associated pathologies are discussed. The use of EP3 antagonist was found to affect blood glucose levels in mice fed a high-fat diet and made insulin deficient by streptozocin injection.

### FIELD OF THE INVENTION

The present invention relates to identification of genes that are associated with Type II Diabetes Mellitus (T2DM), and screening methods to identify chemical compounds that act on those targets for the treatment of T2DM or its associated pathologies.

### BACKGROUND OF THE INVENTION

Type II Diabetes Mellitus (T2DM) affects nearly 8% of adults in the US population and accounts for nearly 10% of all health care dollars spent in the US, and the incidence is steadily increasing—from 1997 to 2002 it rose by 43% among all adults and by 39% on an age-adjusted basis. The incidence of T2DM is increasing in other parts of the world, and it can now considered a worldwide epidemic. It is associated with serious co-morbidities including obesity, hypertension, hyperlipidemia and cardiovascular disease. The major characteristics of T2DM are fasting hyperglycemia and relative insulin insufficiency. Untreated, hyperglycemia can cause long-term microvascular and macrovascular complications, such as nephropathy, neuropathy, retinopathy and atherosclerosis. Insulin resistance has been seen to be a key feature of T2DM for some time (Lillioja et al 1993) and more recently decreased beta cell function has also been identified as an important factor in the early development of the disease (Kahn et al 2003.)

The purpose of the present study was to identify genes coding for tractable targets that are associated with T2DM, develop screening methods to identify compounds that act on the gene products, in order to develop medicines to treat T2DM and its associated pathologies.

### SUMMARY OF THE INVENTION

An aspect of the present invention is a method for screening small molecule compounds for use in treating Type II Diabetes Mellitus (T2DM), by screening a test compound against a target selected from the group consisting of PTGER3, inducible Nitric Oxide Synthase (iNOS) BRAF, Caspase 8, Integrin Alpha 9 (ITGA9), ADAMTS7 (A Disintegrin-like And Metalloprotease domain with Thrombospondin motifs 7) and Amyloid Precursor Protein. Activity against said target indicates the test compound has potential use in treating Type II Diabetes Mellitus.

A further aspect of the present invention is a method of lowering serum blood glucose level in a mammal, such as a human, in need thereof, by administering an EP3 antagonist in an amount effective to decrease serum blood glucose level.

### BRIEF DESCRIPTION OF THE FIGURES

FIG. 1 provides a graphic of the gene analysis process used in the present studies.

FIG. 2 graphs the effects of GW671021 B 100 mg/kg on serum glucose over a 5 hour time course in mice fed a high fat diet (WD). Data represent the average change in blood glucose measurement (mg/dL) in the 10 mice per treatment group.

FIG. 3a graphs the effects of various compounds on serum glucose over a 5 hour time course after a first dose of compound, in mice fed a high fat diet and treated with streptozocin. X-axis represents time in hours, and the Y-axis represents blood glucose (mg/dL) change from baseline.

FIG. 3b graphs the effects, on serum glucose over a 5 hour period, of the same treatments as in FIG. 3a, after the 15^{th} dose of compound (Day 7). X-axis represents time in hours, and the Y-axis represents blood glucose (mg/dL) change from baseline.

FIG. 3c graphs the effects, on serum glucose over a 5 hour period, of the same treatments as in FIGS. 3a and 3b, after the 27^{th} dose of compound (Day 13). X-axis represents time in hours, and the Y-axis represents blood glucose (mg/dL) change from baseline.

FIG. 4 graphs the serum glucose level (single measurement in each of the ten mice in each group) after 14 days of treatment. The first six bars of FIG. 4 represent the same treatment groups as shown in FIGS. 3a-3b; the final bar is a group of mice that did not receive Streptozocin and did not receive any compound (Vehicle only). Blood glucose is provided in mg/dL on the Y-axis.

### DETAILED DESCRIPTION

The present inventors tested genes that encode for potential tractable targets to identify genes that are associated with the occurrence of T2DM, and to provide methods for screening for compounds with potential therapeutic effects in T2DM.

A primary genetic association screen of 1405 genes identified 256 genes as associated (P<0.05) with T2DM in a sample of 401 Caucasian cases and 400 Caucasian controls. These 256 genes were subsequently evaluated for genetic association with T2DM in a second large independent sample of 1166 Caucasian cases and 1260 Caucasian controls. Fifty-three genes (P<0.05 in both primary and secondary screens) were subject to permutation analysis, revealing 21 genes P<0.05. Data simulations suggested that approximately 50% of the 21 genes are likely to be associated with T2DM. Further investigation of the 21 genes was carried out in order to identify those having the strongest rationale for involvement in the development of T2DM.

As used, herein, a ‘tractable target’ or ‘druggable target’ is a biological molecule that is known to be responsive to manipulation by small molecule chemical compounds, e.g., can be activated or inhibited by small molecule chemical compounds. Classes of ‘tractable targets’ include, but are not limited to, 7-transmembrane receptors (7™ receptors), ion channels, nuclear receptors, kinases, proteases and integrins.

Prostaglandin E Receptor (EP3, PTGER3) was identified as a predisposing gene for Type II Diabetes (T2D) (see Example 1). The studies reported herein indicate that EP3, as a potential tractable or druggable target, is a biological molecule that should be responsive to manipulation by small molecule chemical compounds for therapeutic purposes in T2D.

### Example 1

### Subjects and Methods

The primary sample set consisted of 401 Caucasian cases and 400 Caucasian controls. The cases were from a collection of patients who participated in GlaxoSmithKline (GSK) clinical trials. All subjects gave informed consent for the use of their DNA in this study. Selection criteria included age of onset (between 30 and 70 inclusive), Body Mass Index (BMI) less than 35, and at least one relative with T2DM. The controls were selected from a large cohort of approximately 1000 healthy Caucasians from five sites: Australia (61), Canada (87), Germany (83), Switzerland (127) and the US (41). ‘Healthy’ was defined as individuals who are free from clinical cardiac, pulmonary, gastrointestinal, hepatic, renal, haematological, neurological and psychiatric disease as determined by history, physical examination or screening investigations. Summary information for this primary screen population is seen in Table 1.

A total of 1166 Caucasian cases and 1260 Caucasian controls were genotyped in the Secondary screen. The cases were selected from four different regions: Birmingham (Birmingham Heartlands Hospital, UK), Kaiser (California), GENNID (US collection for the American Diabetes Association), and France. The controls were selected from in-house control collections, matched for age and geographical origin. A detailed breakdown of these individuals is seen in Table 2.

### Target Genes

Relatively few human proteins, approximately a hundred in total, are targets for effective small molecule drugs. It was considered reasonable to include all the members of these families for which a sequence was available. At the time some of the genes were not exemplified in the public domain and were discovered through the analysis of expressed sequence tags or genomic sequence using a combination of sequence analysis. In addition, genes were selected because they were the targets of effective drugs even though they were not part of large protein families. Finally disease expertise was employed to select genes whose involvement in diabetes was either proven or suspected. Approximately 1600 genes were selected in total.

### SNP Indentification

The genes were automatically assembled and annotated with a region, 5′, 3′, intron and exon. SNPs were mapped using BLAST to the manually curated genomic sequences and were selected up to 10 kb from the start and stop sites of the transcripts with an average intermarker distance of 20 Kb. SNPs with a minor allele frequency (MAF)>5% were selected. However, all known coding SNPs were included irrespective of MAF. Approximately 10% of genes had fewer than 6 SNPs and these were subjected to SNP discovery using 24 primer pairs per gene to amplify 12 DNAs selected from Coriell Cell Repository of female CEPH cell-line samples. (CEPH refers to the Centre d'Etude du Polymorphisme Humain, which collected Northern European DNA samples.) All of the discovered SNPs were validated using the FAST technology and Amplifluor and one additional SNP per gene was selected for genotyping. A marker selection algorithm was employed to remove highly correlated SNPs to reduced the genotyping requirement while maintaining the genetic information content throughout the regions (Meng et al, 2003).

### Sample Preparation and Genotyping

DNA was isolated from whole blood using a basic salting-out procedure. Samples were arrayed and normalized in water to a standard concentration of 5 ng/ul. Twenty nanogram aliquots of the DNA samples were arrayed into 96-well microtiter plates. For purposes of quality control, 6% of the samples were duplicated on the plates and two negative template control wells received water. The samples were dried and the plates were stored at room temperature until use. Genotyping was performed by a modification of the single base chain extension (SBCE) assay previously described (Taylor et al. 2001). Assays were designed by a GlaxoSmithKline in-house primer design program and then grouped into multiplexes of 50 reactions for PCR and SBCE. Following genotyping, the data was scored using a modification of Spotfire Decision Site Version 7.0 Genotypes passed quality control if: a) duplicate comparisons were concordant, b) negative template controls did not generate genotypes and c) more than 80% of the samples had valid genotypes. Genotypes for assays passing quality control tests were exported to an analysis database.

### Data Handling

The GSK database of record for analysis-ready data is called SubjectLand. This database contains all genotypes, phenotypes (i.e. clinical data), and pedigree information, where applicable, on all subjects used in the analysis of data for these studies. SubjectLand does not maintain information regarding DNA samples, but is closely integrated with the sample tracking system to maintain the connection between subjects and their samples at all times. The analytical tools used in the analysis process described below interface directly with subject data in SubjectLand. This interface also archives the files used in analysis as well as the results.

### Analysis

In both the primary and secondary screens, each marker was examined for deviation from Hardy-Weinberg Equilibrium (HWE) by examining genotype distribution in the controls (female controls for X-linked markers) using a Chi square test. A HWE permutation test was performed when the HWE chi-square P<0.05 and when at least one genotype cell had an expected count <5 (Zaykin et al, 1995). Markers with a HWE P<0.001 were excluded from analysis as were markers monomorphic in cases and controls,

For each individual marker, allelic and genotypic association with disease was assessed using Fisher's exact test (FET) for 2×2 and 2×3 contingency tables. The ‘PROC FREQ’ procedure in the statistical software package SAS was used to do this. PROC FREQ computes exact P-values (significant P_{—}0.01) for general R×C tables using the network algorithm developed by Mehta and Patel (1983). The algorithm provides a substantial feasible only for small sample numbers.

The Composite Haplotype Method was used to test for association between haplotypes and disease (Zaykin et al, in press). Unlike the EM algorithm this does not assume HWE. Haplotypes were constructed by selecting up to five SNPs within a gene, or fewer than five SNPs if a gene contained four or fewer SNPs. The number of possible haplotypes with a window of n markers is 2ⁿ. For example, if a window of three biallelic markers was used, eight different haplotypes are possible (although fewer may be observed). The global haplotypic test contrasted the frequencies of all observed haplotypes between cases and controls in a single test. In addition, separate tests are computed to compare each of the eight individual haplotypes between cases and controls. Evidence of association based on the global test was examined for this study.

Distribution of P-values from the allelic, genotypic and haplotypic tests was examined. The number of tests with P-values below a series of cut-points was evaluated to determine whether this number of tests was inflated with respect to what would be expected under the null distribution of no association. (The probability of ≧m observed number of significant tests out of n total tests at a cut-point p was calculated using the binomial probability.) Inflation in the number of observed significant tests over a range of cut-points, suggests that the case and control groups are not well matched. The inflated number of positive tests may be due to population stratification rather than to association between the associated SNPs and disease.

When this general inflation was observed, we adjusted the test P-values, using a Genomic control approach. Devlin et al (2001) proposed dividing the tests for association by \hat{λ}
The simplest implementation suggested is to estimate \hat{λ} as follows
\hat{λ}={median (S_{c+1},S_{c+2}, . . . , Sₙ)/0.456} where S refers to the chi-square P-value or FET P-value.

In this study some small genes had only one to two SNPs analyzed whereas some of the larger genes, such as ion channels, had more than 25 SNPs analyzed. The greater the number of SNPs and tests performed on a gene, the more likely it is to appear significant by chance alone. To account and correct for the variable number of tests conducted across genes, a gene-based permutation test described previously (Roses et al.) was applied. All the SNPs within the gene were analyzed using allelic, genotypic and haplotypic association tests, and a gene with an empirical P≦0.05 was considered “replicated” as associated with disease.

### Results

In the primary screen following quality control measures 325 SNPs were excluded as they were monomorphic in both cases and controls, and 55 SNPs were excluded for HWE deviation (P<0.001), leaving 4267 SNPs mapping within 1405 genes for analysis (Table 3).

Examination of the P-values distribution revealed evidence of population stratification. Adjustments to the P-values were made using the genomic control approach of Devlin and Roeder (2002). The value for lambda was 1.10 for the genotypic test and 1.08 in the allelic test. After this adjustment a subset of genes with a SNP or haplotype genetically associated with T2DM (P<0.05) was selected for genotyping in the secondary association screen. Not all SNPs in these genes were put included, only SNPs or haplotypes with P<0.05 together with other SNPs from those same genes with a P<0.2.

In the secondary screen, 852 SNPs mapping within 257 genes were genotyped. Two monomorphic SNPs and five SNPs with HWE P<0.001 were excluded from analysis, resulting in analysis of 845 SNPs mapping within 256 genes. Only one gene was excluded from the analysis following this Quality Control. There was an average of 3.3 SNPs per gene.

Population stratification was again observed after examining the distribution of the test P-values. An adjustment was made to the raw P-values, using the same genomic control approach as above. The value for lambda in this screen was 1.06 for the genotypic test and 1.17 in the allelic test.

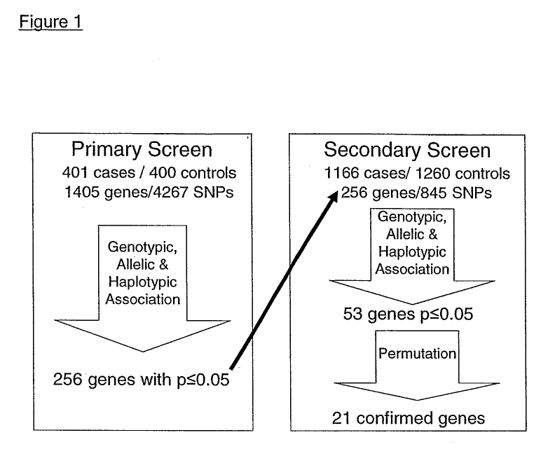

We observed 53 genes with an adjusted P<0.05 in both the primary and secondary screens. These genes were then assessed by permutation analysis. The overall analysis process is summarised in FIG. 1 below.

The genes assessed in the permutation analysis are listed in Table 4 Detailed results from both the primary and secondary screens for genes with a permutation P<0.05 are shown in Table 5.

### Discussion

Twenty one genes were identified as genetically associated with T2DM following the permutation analysis. However, as will be apparent to one of skill in the art, it is possible that some of these associations are false positives. From the results of simulation tests, described previously (Roses A D, Burns D K, Chissoe S, Middleton L, St. Jean P. Disease-specific target selection: a critical first step down the right road. Drug Discovery Today 2005; 10:177-189) it is possible that approximately 50% of the confirmed genes may be false positives.

Six genes (BRAF, ITGA9, NOS2A, CASP8, ADAMTS7 and APP) had the same SNP(s) associated in the primary and secondary screen, as well as same test in the same direction (i.e. same suspect allele or genotype.) A further four genes (ESPL1, MAP3K9, CHRNA1 and ROR1) had the same SNP and same test associated in the primary and secondary screen, but in a different direction (i.e. different alleles or genotypes).

Discussion of the biological roles of certain genes are provided below.

NOS2A (iNOS)

The Nitric Oxide Synthase family encodes for three isoforms (1, 2, and 3) that all produce nitric oxide as a byproduct of the conversion of arginine to citrulline. Nos2A or inducible Nos (iNOS) was first identified in macrophages via its up-regulation in response to cytokine combinations (e.g. TNF, IL1, IFNg) or LPS but has also been shown to play important roles in other tissues most notably muscle, liver, and pancreas. Tissue macrophage numbers have been positively correlated with adiposity and are a major contributor to iNOS in this tissue (Weisberg et al., 2003). Inflammatory cytokines produced from adipocytes and inflammatory cells have been shown to mediate insulin resistance (Shoelson et al, 2001). Nos2A induction and NO production as a result of cytokine and LPS treatment mediates effects consistent with insulin resistance including inhibition of insulin stimulated glucose uptake and decreased Glut 4 translocation (Bedard et al., 1997; Balon et al., 1994, 1997; Kapur et al., 1997).

Consistent with these observations the NOS2A knockout mouse has enlarged epidydimal fat stores but decreased muscle insulin resistance in diet-induced obesity (Perrault and Marett, 2001). In pancreas, NOS2A induction and NO production have also been associated with down regulation of insulin secretion (Corbett et al., 1993) and fatty acid and interleukin-1 dependents beta cell apoptosis (Shimabukuro et al., 1998; Todaro et al., 2003). In vivo nicotinamide or aminoguanidine (non-selective iNOS inhibitors) prevented iNOS expression in islets and decreased beta cell dysfunction while blocking beta cell destruction and hyperglycemia (Shimabukuro et al., 1997). Likewise a more selective iNOS inhibitor, L-NIL, ameliorated fasting hyperglycemia and hyperinsulinemia in male ob/ob mice in part through improved hepatic insulin receptor signalling (Fujimoto et al. 2004).

### Prostaglandin E Receptor (EP3, PTGER3)

PTGER3 receptor ligands are known to play a role in adipose and liver biology. However it is unknown whether the polymorphism associated herein with T2DM leads to activation or inactivation of the receptor and this cannot be decided based on current knowledge of how the receptor affects glucose homeostasis. PTGER3 (EP3) is the most widely distributed of the four subtypes of prostaglandin receptors. There are nine different splice variants of EP3, all generated from one gene. One splice variant is in the 3′-untranslated region; the eight expressed isoforms differ most significantly in their carboxyl-terminal domains. All eight isoforms exhibit similar ligand binding properties (PGE₂=PGE₁>>PGF₂ₐ>PGD₂, for EP_{3-I}, EP_{3-II} and EP_{3-III}). Different isoforms, when expressed in heterologous systems in vitro, can couple to different G proteins and thus activate a variety of signal transduction pathways, but most isoforms couple to Gi in vivo (Adam et al., 1994; Kotani et al., 1995; Kotani et al., 1997; Namba et al., 1993).

EP3 receptors are expressed in adipose tissue and liver (unpublished data), and EP3 ligands have several distinct effects on these tissues that synergize with insulin's effects to promote glucose uptake and incorporation into fat stores (adipose tissue) and glycogen (liver), and minimize their utilization. PGE2 is a well-known antilipolytic agent, and this effect is mediated by EP3 (Richelsen et al., 1984, Richelsen and Beck-Nielsen, 1984). Additionally, both PGE2 and PGE1, acting via EP3, attenuate suppression of insulin-stimulated glucose uptake by adrenergic ligands and other counter regulatory agents (Kuroda et al., 1987), and PGE2 suppresses adrenergic-stimulated respiration in brown adipose tissue. Finally, PGE2 itself stimulates glucose uptake in isolated adipocytes (Richelsen, et al., 1985) via a process that may be mediated by both Gi and the rho-kinase pathways (Hasegawa et al., 1997; Thomson et al., 1997; van den Berghe et al., 1996; Shum et al., 2003; Chiang et al., 2003). In the liver, EP3 similarly has insulin-mimetic effects on glycogen and lipid metabolism: PGE2 inhibits glucagon-mediated stimulation of glycogenolysis (Puschel G P, et al.), and suppresses the expression of lipogenic genes such as fatty acid synthase and spot 14 (Mater M K, et al.).

As mentioned, EP3 ligands synergize with insulin to countermand the effects of catabolic agents at the level of both adipose tissue and liver, and thus defects in PGE2 signal transduction, via defects in the EP3 receptor, could result in or contribute to disregulation of energy homeostasis leading to T2DM. Additionally, the EP3 receptor may also be involved in mediating insulin resistance and interference with insulin secretion. Kurihara et al demonstrated that the EP3 receptor is involved in the induction of IL-6 (Kurihara et al.). IL-6 is one of several cytokines that elicits insulin resistance in hepatocytes (Senn et al. 2002) and adipocytes (Rotter et al. 2003), although its role in whole-body insulin resistance has been questioned recently (Carey et al. 2004). Further, Tran et al. have demonstrated that PGE2 inhibits insulin secretion in response to elevations of IL-1 and that sodium salicylate reverses this effect (Tran et al., 1999, 2002).

From the foregoing it is not clear whether the T2DM-associated polymorphism in EP3 activates or inhibits receptor function. For this reason the most intriguing insight is that the EP3 receptor mediates the effects of the cannabinoid receptor, CB-1 (Yamaguchi et al.). CB-1 receptor antagonists have been reported to elicit weight loss and improve glucose tolerance and insulin sensitivity in experimental animals and humans.

### Endothelin-1 (EDN1)

The present study implicates defects in endothelin-1 action or amounts of the endothelin-1 polypeptide as a causal agent for T2DM. Reports in the literature discuss endothelin-1 in the context of insulin resistance associated with T2DM, obesity and the Metabolic Syndrome (see, for instance, Mather et al., Juan et al. 1996, Ottoson-Seeberger et al., Juan et al. 1998). Endothelin-1 is a small (21 amino acid) peptide produced by endothelial cells. It exerts potent vasoconstrictor effects on vascular smooth muscle via either of two 7-TM receptors, ET_{A} and ETB (Yanagisawa et al., Inoue et al., Sakurai et al.). To understand how ET-1 action at the level of the endothelium might play a causal role in the development of T2DM, recall that insulin sensitivity is a complex biological property that critically depends on three variables: insulin responsiveness of the target tissue, access of insulin to the cells of that tissue, and also, blood flow (Baron). ET-1 affects all three processes. The rate of blood flow is controlled, in part, by endothelium-dependent vasodilatation, which is blunted in insulin resistant states including T2DM and obesity (Steinberg et al.). Because endothelium-independent vasodilation is unaffected in these subjects, the defect is likely due to a polymorphism in the ET-1 gene that either suppresses ET-1 activity or leads to faulty processing and/or secretion of ET-1 from the endothelium. The present results support the former possibility.

In addition to its indirect influence on insulin action via vasodilatation, ET-1 also has complex direct effects on the insulin responsiveness of adipose tissue. Normal insulin responsiveness depends on a balanced, appropriate supply of insulin, and ET-1 potentiates insulin secretion (Gregersen et al.; Brock et al.). On the other hand, while maintaining adequate stores of insulin-responsive adipose is a sine qua non for normoglycemia, chronic exposure to ET-1 disrupts adipose function and development. Acute (30 minute) treatments of cultured 3T3L1 adipocytes with ET-1 enhanced glucose uptake, by promoting translocation of the insulin-sensitive glucose transporter, GLUT4, to the surface membrane (Wu-Wong et al., Imamura et al.). The ET_{A} receptor-specific antagonist A-216546 blocked this effect (Wu-Wong et al.), but in addition Olefsky's group showed that G□_{q/11} coupled the ET_{A}-generated signal to PI(3)-kinase (Imamura et al.). By contrast, chronic (>2 hrs) endothelin-1 treatment of adipocytes lead to desensitization of the insulin signaling pathway, and this effect was also mediated by the ET_{A} receptor (Lee Y-C et al., Ishibashi et al.). Therefore, it is not unexpected that a polymorphism in the ET-1 gene that would disrupt its normal synthesis/release and/or its interactions with the ET_{A} receptor would have profound effects on glucose homeostasis. Furthermore, ET-1 has a similar biphasic effect on adiponectin secretion from cultured adipocytes (stimulating acutely but inhibiting during chronic treatment; Clarke et al.). Adiponectin is a 30 kD polypeptide secreted exclusively by adipose tissue that plays an important, albeit still incompletely defined, role in maintaining whole-body insulin sensitivity (Yamanchi et al, 2001). Moreover, chronic exposure to ET-1 blocks differentiation of human preadipocytes (Hauner et al.). Therefore, disturbances in ET-1 function could significantly affect insulin action and energy homeostasis by antagonizing insulin's effects at its target tissue, including the synthesis/release of adiponectin, a major hormone that regulates insulin sensitivity, by controlling access of insulin to its target tissue, and by directly interfering with insulin's effects on its target tissue.

### Vasoactive Intestinal Polypeptide (VIP)

VIP, a member of the glucagon/secretin family, is a 28-aa peptide which is generated from a larger (170 aa) protein by proteolytic cleavage (Itoh et al.). It is widely distributed in peripheral tissue and in the peripheral and central nervous system, and exerts multiple effects: it induces smooth muscle relaxation, it elicits secretion of some hormones, including many counter-regulatory factors, but inhibits the secretion of others, and it modulates the activity of the immune system (Watson et al.). There are two structurally distinct receptors that bind VIP and pituitary adenylate cyclase activating polypeptide (PACAP), PACAP/VIPR-1 and PACAP/VIPR-2. Both types are found in tissue that play important roles in maintaining glucose homeostasis, including adipose tissue, heart and brain, and both receptors are coupled to adenylate cyclase and the phosphatidylinositol/calcium pathways (Wei and Mojsov).

VIP signaling is important for the proliferation and survival of immature pancreatic epithelial cells. VPAC2 is expressed in E13-stage embryos in PDX-1-positive cells, and VIP treatment of pancreatic epithelia resulted in decreased apoptosis and increased cell proliferation (Rachdi et al.). This may be one factor in regulating the number of cells that mature, insulin-secreting cells.

In the adult, VIP is synthesized and released by the hypothalamus and pituitary, and is involved in regulating pituitary function, including stimulating the release of growth hormone (GH), ACTH and prolactin and inhibiting the release of somatostatin (Fahrenkrug and Emson). Because GH, ACTH and somatostatin all modulate whole-body insulin sensitivity (Wilson et al.), a link between T2DM and VIP may be straightforward. Growth hormone exerts both insulin mimetic and counter-regulatory effects on glucose disposal and, in high concentrations, elicits insulin resistance. Glucocorticoids are also potent counter-regulatory hormones, and by promoting glucocorticoid biosynthesis ACTH also has a significant impact on insulin resistance. Somatostatin blocks insulin release from pancreatic □-cells and glucagon secretion from □-cells, and therefore indirectly affects both glucose disposal and endogenous glucose production. VIP may also be considered a counter-regulatory hormone itself because it stimulates lipolysis in adipose tissue and glycogenolysis in liver. By promoting the synthesis/release of this triad of hormones, and through its own direct activities, VIP can have a significant influence on whole body insulin sensitivity and glucose homeostasis. Moreover, VIP also induces smooth muscle relaxation, and therefore could be involved in controlling the relaxation of small blood vessels associated with increasing blood flow needed for insulin action, as described above for ET-1.

VIP also affects cytokine synthesis/release from immune cells, including macrophages. An emerging area of intense interest in the diabetes community is the connection between inflammation and T2DM (Duncan et al.). Inflammatory genes are upregulated in adipose of T2DM subjects (Shang et al.). Macrophages populate adipose tissues and are a major source of the cytokines that interfere with insulin action and block adipocyte differentiation and adipose function, including TNF□, IL-6 and IL-8. VIP inhibits TNF□ and IL-8 production by macrophages (Delgado et al., Delgado and Ganea). The present results suggest that defects in VIP synthesis/release or function may contribute to the enhanced release of proinflammatory cytokines associated with T2DM.

### MAP3K9 (MLK1):

Mixed lineage kinases are cytokine activated serine/threonine protein kinases that are implicated in the control of apoptosis. In neuronal cells, overexpression of MLKs elicits apoptosis while dominant-negative versions or pan-MLK inhibitors block apoptosis due to NGF withdrawal (Harris et al. 2001, Mielke and Herdegen, 2002; Xu et al. 2001). Interestingly NGF and its receptors are expressed in cultured islet cells and NGF neutralization induces apoptosis (Pierucci D. et al., 2001). MLKs participate in a signal transduction cascade between Cdc42/Rac1 and MKK4/7 that ultimately regulates c-Jun amino-terminal kinase (JNK) and the p38 mitogen-activated protein kinase (MAPK) pathways (Gallo and Johnson, 2002). The JNK1 knockout has been shown to have improved insulin sensitivity (Hirosumi, 2002). Although untested, MLK1 as an upstream activator of JNK1 could likewise mediate insulin resistance. Recently MLK1 has been implicated in beta cell line maturation and is expressed in the embryonic pancreas (DeAizpurua et al., 1997). MLK3 has been shown to regulate ERK signaling through the activation of B-RAF (Chadee and Kyriakis, 2004). Whether MLK1 could play an identical role in activation of ERK is not known. Since MLK1 is not significantly expressed in peripheral tissues it is more likely that the current genetic results point to a contribution of MLK1 to the □-cell degeneration that occurs in the later stages of T2DM.

### B-Raf:

Several molecules that are modulators of Ras (both upstream (IGF-1, IRS-2) and downstream modulators (AKT, p70sK1)) have been implicated in □-cell proliferation (de Mora et. al., 2003). The raf genes encode for a family of cytoplasmic serine/threonine kinases (A-raf, B-raf and C-raf) that are activated in response to a variety of extracellular stimuli such as insulin, nerve growth factor (NGF), platelet derived-growth factor (PDGF) and are important mediators of signals involving cell growth, transformation and differentiation (Williams and Roberts, 1994; Kolch 2001). Rafs participate in a signal transduction cascade between Ras and MEK1/2 with B-Raf playing a prominent role in activation of the ultimate effector ERK1/2 (O'Neill and Koch, 2004). B-Raf is also activated by glucose-dependent insulinotropic polypeptide (GIP) via a cyclic AMP dependent protein kinase/Rap1 mediated pathway (Ehses et al., 2002). Genetic and biochemical approaches have identified Rafs as mediators of cell survival although effects appear to be cell/tissue specific (O'Neill and Kolch, 2004). Mice with a targeted disruption in the B-Raf gene die of vascular defects during mid-gestation due to apoptotic death of differentiated endothelial cells (Wojnowski et al., 1997). The role of ERKs in signaling and apoptosis are complex and may depend on the particular upstream pathway as well as its duration and kinetics (O'Neill and Koch, 2004). The anti-apoptotic activity of insulin is in part due to activation of ERK (Kang et al. 2003). Cytokines may contribute to beta-cell apoptosis in the early stages of type 1 diabetes mellitus. IL-1 beta induces activation of both p38 and ERK1/2 and ERK1/2 inhibition partially blocks the apoptotic effects of IL-1 in primary beta-cells (Pavlovic et. al, 2000).

### Caspase 8

Apoptosis is a regulated cell death program controlled by extrinsic and intrinsic signaling pathways. The intrinsic pathway involves stress signals that activate pro-apoptotic members of the Bcl-2 family, inducing permeabilization of mitochondria and release of apoptogenic factors. The extrinsic pathway is mediated by death receptor members of the TNF receptor superfamily (eg. TNFR1, TRAIL, and FAS). Chronic cytokine production as a result of obesity is a recently observed feature of type II diabetes and cytokines have been shown to contribute to beta cell apoptosis (Mandrup-Poulsen, 2003). Receptor-mediated cell death is mediated by the recruitment of adaptor proteins which then bind to procaspase to generate a death inducing signaling complex that leads to activation of the initiator caspase-8. Caspase-8 in turn cleaves and activates caspase-3 which executes apoptosis. Human islets normally express Fas ligand but not the receptor and high glucose can upregulate Fas receptor coincident with the induction of apoptosis (Maedler et al., 2001). Overexpression of cFLIP (Fas-associated death domain-like interleukin-1beta-converting enzyme-inhibitory protein) can prevent activation of this caspase cascade and rescue beta cells from apoptosis (Cottet et al., 2002; Maedler et al. 2001). The genetic association shown here in two independent populations of diabetics suggests that polymorphisms in the Caspase 8 gene may tip the balance towards □-cell destruction.

### Amyloid Precursor Protein (APP) Beta

Type II diabetes is characterized by □-cell loss and islet localized amyloidosis. In beta cells amyloid polypeptide (IAPP) is normally co-secreted with insulin into the circulation following cleavage by prohormone convertases (Johnson et al., 1992). Alzheimer disease is characterized by the loss of neocortical neurons and focal amyloid deposits. The amyloid deposits formed in Alzheimer disease are formed by cleavage of A□ by □-, □-, and □-secretases and is released as insoluble fragments into the extracellular space. Amyloid fibrils in each disease may result from altered secretion and or processing of the precursor proteins and have been shown to be toxic in several cell models. A transgenic mouse model of AD in which A□ is overexpressed actually develops widespread pancreatic acinar amyloid deposits (without brain amyloidosis) although does not develop the hyperglycemia seen in the IAPP transgenic (Kawarabayashi et al. 1996; Vechere et al. 1996). Proteomic studies have shown that proteins implicated in Alzheimer disease are highly expressed in normal pancreatic islets (Nicolls et al, 2002). APP as well as certain of the cleavage enzymes are expressed in beta cells (Figueroa et al., 2001) although there is no reported evidence that these proteases play a role in pancreatic amyloidosis. Alzheimer's disease and type 11 diabetes are often found in same patients and AD patients may have more amyloidosis in their islets (Leibson et al, 21997; Janson et al., 2004) suggesting activation of common pathways. In support, JIP1b, an essential scaffold protein involved in JNK signaling, binds APP and facilitates JNK phosphorylation of APP to mediate it's association with kinesin proteins (Inamota, 2003). Likewise fibrillogenic amylin has been shown to activate JNK1 (Liu et al., 2003).

### Gamma Amino Butyric Acid Receptors Beta1 and Gamma2 (GABARB2, GABARG2)

GABA is the major inhibitory neurotransmitter of the nervous system. There are three GABA receptors, the A and C receptors are multisubunit ion channels while the B family are 7-transmembrane receptors. Sixteen human GABAA receptor cDNA have been cloned. GABAA receptors assembled from 5 GABAA subunits arrange to form an ion channel (Chebib and Johnston, 1999). The most common GABAA receptor in the CNS consists of 2 copies of □1 and □2 and 1 copy of □2. GABA is present in pancreatic □-cells at concentrations comparable to CNS whereas GABAA receptors are confined to pancreatic □- and □-cells. GABA released from □-cells, binds to GABAA receptors on □-cells to inhibit the secretion of somatostatin and glucagon from □-cells therefore, stimulating insulin release (Park and Park, 2000; Wendt et al., 2004). The GABAA receptor in the human pancreas expresses the □2, □3, and □1 transcripts (Yang et al., 1994) but not the □1 and □2 transcripts suggesting that the genetic association reported here is in receptors that are mediating central rather than peripheral effects. Interestingly, deletion of CB-1 R in mice (which prevents diet-induced obesity) results in increased anxiety-like behaviours that are not modified by the GABAA receptor agonist bromazepam suggesting that deletion of CB-1R may alter GABA signalling or functionally disrupt GABAA receptor subunit structure (Uriguen et al., 2004).

### Integrin Alpha 9 (ITGA9)

Integrin □9 is an adhesion molecule that is expressed in smooth muscle, skeletal muscle and liver, as well as in airway epithelium and squamous epithelium. It is known that cells in mature tissue depend on the appropriate cell-cell contacts to develop and maintain their differentiated phenotype (Trinkaus; Kuhn). However, the expression of genes associated with mature tissue is decreased in both adipose tissue and skeletal muscle from diabetic individuals (Nadler et al., Patti et al., Mootha et al.). It thus appears that cells in these key tissue from diabetics may be unable to maintain their fully differentiated phenotype. Because the spatial and temporal distribution of ITGA9 suggests that it plays a role in maturation and/or maintenance of the differentiated phenotype, and not in primordial organ formation, the disease-associated polymorphism uncovered in the present HitDip study may either alter the level of expression of ITGA9, or lead to a malfunctioning protein. Either one of these outcomes could lead to a functional dedifferentiation, or under-differentiation, of organs such as liver and skeletal muscle which, in turn, would have a negative effect on glucose homeostasis and insulin sensitivity.

### Example 2

### EP3 Antagonist Effect on Glucose

The studies reported below dosed male C57BL/6J mice with an EP3 antagonist, GW671021B, and monitored the effects on glucose homeostasis by measuring tail-vein blood glucose. The animals were either (a) fed a high fat diet to induce mild insulin resistance, or (b) were fed the high fat diet and also treated with a low dose of streptozotocin (which damages the pancreatic beta-cells) resulting in both insulin resistance and insulin deficiency (as in T2DM patients).

Materials and Methods: Male 6 week old C57BL/6J mice (Jackson Laboratory, Bar Harbor Me.) were made mildly insulin resistant by feeding a high fat Western Diet (WD) (Research Diets, D12079B) for 3 weeks. Test mice were then made insulin deficient by intraperitoneal injection (IP) with 100 mg/kg Streptozotocin (STZ or streptozocin; Zanosar®, Sicor Pharmaceuticals, Irvine, Calif.) reconstituted in citric acid. Control mice (also on the WD) were injected with the same volume and concentration of citric acid without the STZ. Two weeks after the STZ (or control citric acid) injections the mice received injections of either GW021 in vehicle, or vehicle alone (as described below). Student's t-test or Tukey's HSD (Honestly Significant Different) test was used to determine the significance of the results.

Compound GW671021 (also referred to herein as GW021 or '021) is an EP3 antagonist described as compound 9 in Juteau et al., Bioorg. Med. Chem. 9:1977-1984 (2001). GW671021B is the sodium salt.

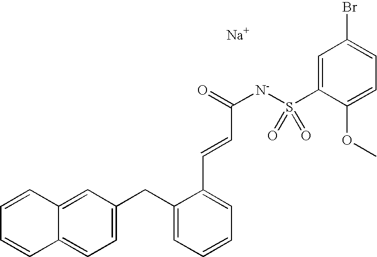

Acute Glucose Lowering Study: At 6:30 am mice were transferred to clean cages with water in the procedure room and allowed to acclimate for 1 hour. After acclimation, a small snip was made at the tip of the tail for blood collection and baseline glucose measurements were collected using the Bayer Elite XL glucometer. Mice were dosed with either compound GW671021B (100 mg/kg body weight) in a suspension in 0.5% HPMC, 0.1% Tween 80; or dosed with Vehicle only (HPMC, 0.1% Tween 80). Whole blood for glucose measurement was collected at 0.5, 1, 2, 3, 4, and 5 hours after dosing. Mice did not have access to food after dosing. There were 10 mice in each treatment group. Results are shown in Table 6.

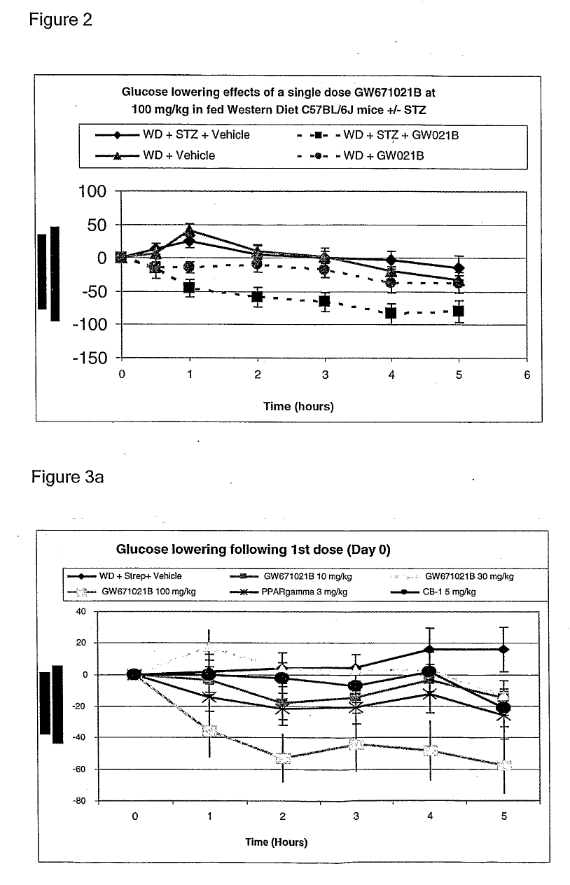

Results: Table 6 shows, in mice fed a high fat Western Diet (WD), the acute effects of GW671021B 100 mg/kg on serum glucose over a 5 hour time course. Data represent an average blood glucose measurement (mg/dL) of the 10 mice in each treatment group at each time point. The data are displayed graphically in FIG. 2.

TABLE 6
Acute effects of GW671021B on Glucose

| | | | | | | | |
|---|---|---|---|---|---|---|---|
| N = | 0 hrs | 0.5 hrs | 1 hr | 2 hrs | 3 hrs | 4 hrs | 5 hrs |

STZ-treated groups

| | | | | | | | | |
|---|---|---|---|---|---|---|---|---|
| WD + STZ + Vehicle | 10 | 0 | 13 | 24 | 5 | 0 | −3 | −15 |
| (no GW021) | | | | | | | | |
| WD + STZ + | 10 | 0 | −16 | −46 | −59 | −66 | −83 | −79 |
| GW021B | | | | | | | | |
| (100 mg/kg GW021) | | | | | | | | |

Control Groups
(no STZ)

| | | | | | | | | |
|---|---|---|---|---|---|---|---|---|
| WD + citric acid + | 10 | 0 | 8 | 41 | 10 | 1 | −19 | −33 |
| Vehicle | | | | | | | | |
| (no STZ, no GW021) | | | | | | | | |
| WD + citric acid + | 10 | 0 | −14 | −14 | −9 | −18 | −37 | −37 |
| GW021B | | | | | | | | |
| (no STZ, 100 mg/kg | | | | | | | | |
| GW021) | | | | | | | | |

### EXAMPLE 3

### 14 Day Chronic Study

A 14 day chronic study in C57BL/6J mice was performed. The animals were fed a high fat diet (Western Diet) and treated with a low dose of streptozocin (which damages the pancreatic beta-cells) resulting in both insulin resistance and insulin deficiency (as in T2DM patients). Mice were prepared as generally described above in Example 2. Two weeks after injection of streptozocin, a twice daily dosing schedule was started with:
1. GW67021B (10 mg/kg, 30 mg/kg, or 100 mg/kg); or
2. CB-1 (Rimonabant, antagonist of the CB-1 receptor; commonly used as a positive control for weight-loss inducing drugs) at 5 mg/kg; or
3. GW34784 (PPAR gamma agonist compound as another positive control) at 3 mg/kg (also referred to as GW845 or GW7845))

GW845 (GW347845) is a PPAR-gamma agonist and was used as a positive control for effects on blood glucose in this study. See U.S. Pat. No. 6,294,580 and Yang et al., BMC Pharmacology 4(1):23 (2004).

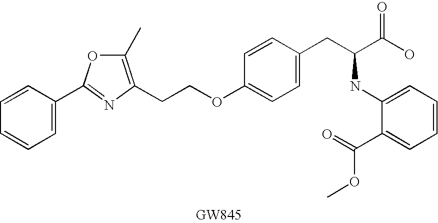

CB-1 (Rimonabant; SR141716) is a selective cannabinoid CB-1 receptor antagonist used as a control in these studies. See Rinaldi-Carmona et al., Febs Lett 350:240-244 (1994); Despres et al., NEJM 353:2121-34; Van Gaal et al., Lancet, 365:1389 (2005).

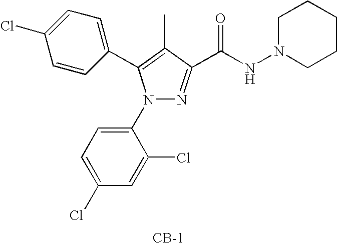

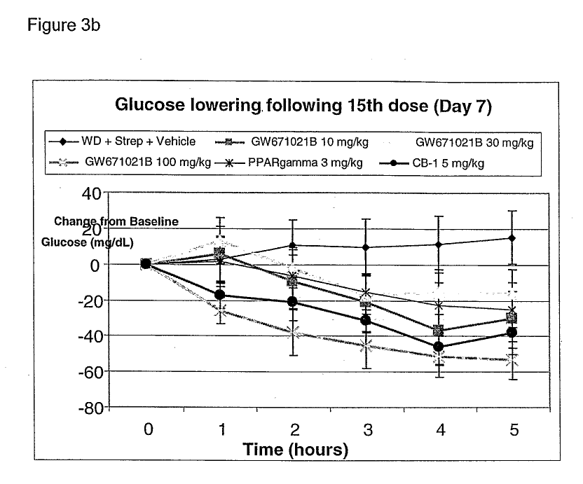

Six groups of mice (N=10 in each group) were used as shown in Table 7. All compounds were dosed in suspension in 0.5% HPMC; 0.1% Tween 80 by body weight. The same general procedures used in the acute study (Example 2) were utilized in this chronic study. Glucose was measured following the 1^{st}, 15^{th} and 27^{th} dose (day 0, 7, and 13) at 0, 1, 2, 3, 4, and 5 hour time points. Results are graphed in FIGS. 3a-3c for the groups of mice receiving WD+Streptozocin+Vehicle (no compound); WD+Streptozocin+GW021 10 mg/kg; WD+Streptozocin+GW021 30 mg/kg; WD+Streptozocin+GW021 100 mg/kg; WD+Streptozocin+GW845 3 mg/kg; WD+Streptozocin+CB-1 5 mg/kg.

TABLE 7

| | Streptozocin | | | | |
|---|---|---|---|---|---|
| | Or | Treatment | | | |
| | Citric Acid | | GW845 | CB-1 | No. of |
| Diet | Control | GW021 | (PPARg) | (Cannabinoid) | mice |
| WD | Streptozocin | — | — | — | 10 |
| WD | Streptozocin | 10 mg/kg | — | — | 10 |
| WD | Streptozocin | 30 mg/kg | — | — | 10 |
| WD | Streptozocin | 100 mg/kg | — | — | 10 |
| WD | Streptozocin | — | 3 mg/kg | — | 10 |
| WD | Streptozocin | — | — | 5 mg/kg | 10 |

### Example 4

### Final Glucose Measurement

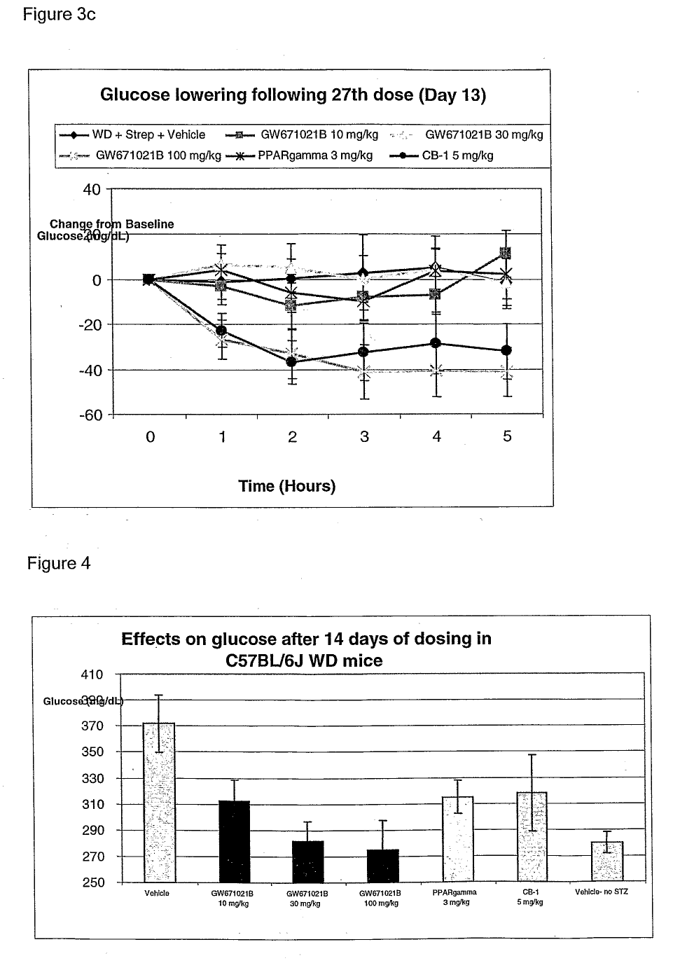

Groups of mice were maintained on diet and dosing schedules as described, and on the 14^{th} day were moved to the procedure room, dosed with a final 28^{th} dose (of compound or vehicle only, according to their group as shown in FIG. 4), then acclimated for about 1 hour with access to food and water before bleeding to measure blood glucose. Results are shown in FIG. 4.

In FIG. 4 the first six bars (from left to right) represent mice that were treated with STZ and vehicle only; STZ and GW021B 10 mg/kg; STZ and GW021 B 30 mg/kg; STZ and GW021 B 100 mg/kg; STZ and GW845 3 mg/kg. The seventh bar represents mice that were not treated with STZ (citric acid control) and were not treated with any compound (Vehicle only). “Vehicle” mice were dosed with HPMC/Tween only (no GW021, CB-1 or GW 845). Each bar represents an average of a single blood draw taken on day 14 from each of the ten mice in a group.

GW671021B had a blood glucose lowering effect (change compared to baseline) in streptozocin-treated C57BL/6J mice fed a high-fat Western Diet over a 5 hour time course (Example 1). GW671021B continued to have glucose lowering effects (change compared to baseline) over 0, 7, and 13 days of treatment in C57BL/6J mice fed a high-fat Western Diet compared to STZ treated mice who did not receive GW671021B (Example 2). As shown in FIG. 4, streptozocin-treated mice fed a high-fat Western Diet who received GW671021B had lower blood glucose levels on the fourteenth day of treatment compared to streptozocin treated mice who were fed a high-fat Western Diet but were not treated with GW671021B.

TABLE 1
Primary Screen Population

| | Cases | Controls |
|---|---|---|
| All subjects | | |
| Caucasian | 401 | 400 |
| Male:Female | 237:164 | 216:183 (1 unknown) |
| Age at diagnosis (std dev) | 51.8 (9.1) | N/A |
| BMI (std dev) | 29.1 (3.2) | N/A |
| Males | | |
| Age at diagnosis (std dev) | 50.9 (9.0) | N/A |
| BMI (std dev) | 29.1 (3.1) | |
| Females | | |
| Age at diagnosis (std dev) | 53.1 (9.1) | N/A |
| BMI (std dev) | 29.1 (3.4) | |

TABLE 2
Secondary Screen Population

| | Cases | Controls |
|---|---|---|
| All subjects | 1166 | 1260 |
| Caucasian | 1166 | 1260 |
| Male:Female | 653:513 | 651:609 |
| Age at diagnosis (std dev) | 51.1 (10.1) | N/A |
| BMI (std dev) | 29.9 (5.8) | N/A |
| Males | | |
| Age at diagnosis (std dev) | 51.1 (9.87) | N/A |
| BMI (std dev) | 29.0 (4.49) | N/A |
| Females | | |
| Age at diagnosis (std dev) | 51.1 (10.4) | N/A |
| BMI (std dev) | 31.0 (6.99) | N/A |

TABLE 3
Summary Statistics for Genes and SNPs analysed

| | Primary | Secondary |
|---|---|---|
| | Screen | Screen |
| Number of SNPs removed from the | | |
| analysis due to; | | |
| Hardy-Weinberg Disequilibrium | 55 | 5 |
| (p < 0.001 in controls) | | |
| Being monomorphic in cases and | 325 | 2 |
| controls: | | |
| Total number of genes not analyzed due | 32 | 1 |
| to QC problems: | | |
| SNPs analyzed for association | | |
| with disease | | |
| Total number of SNPs analyzed: | 4267 | 845 |
| Total number of genes analyzed: | 1405 | 256 |
| Total number of autosomal genes: | 1350 | 242 |
| Total number of X-linked genes: | 55 | 14 |
| Mean number of SNPs per gene: | 3 | 3.3 |
| Range of number of SNPs per gene: | 1-29 | 1-20 |

TABLE 4
Genes tested in the permutation analysis and resultant P-value

| | Permutation | |
|---|---|---|
| | P-value for | Test of |
| Gene | gene | association |
| BRAF | 0.0042 | Allele |
| ITGA9 | 0.0062 | Allele |
| CHRNA1 | 0.0080 | Allele |
| CNR1 | 0.0084 | CHM |
| ADAMTS7 | 0.0090 | CHM |
| GABRB2 | 0.0098 | Genotype |
| ESPL1 | 0.0156 | Allele |
| CASP8 | 0.0172 | CHM |
| MAP3K9 | 0.0186 | Allele |
| ADAMTS17 | 0.0214 | Genotype |
| NOS2A | 0.0248 | CHM |
| ROR1 | 0.0250 | Genotype |
| GABRG2 | 0.0256 | Allele |
| APP | 0.0274 | Allele |
| PTGER3 | 0.0280 | CHM |
| EDN1 | 0.0316 | Genotype |
| ITPR2 | 0.0338 | Genotype |
| ACCN1 | 0.0350 | Allele |
| USP9X | 0.0392 | Genotype |
| VIP | 0.0442 | Genotype |
| ADAM19 | 0.0490 | Allele |
| SRA1 | 0.0554 | Genotype |
| RARA | 0.0560 | Genotype |
| CPVL | 0.0590 | Genotype |
| ITK | 0.0620 | Allele |
| LOC146545 | 0.0700 | CHM |
| ADAMTS2 | 0.0700 | Allele |
| GNAS | 0.0720 | Allele |
| GRM7 | 0.0760 | CHM |
| NTS | 0.0770 | CHM |
| TIF1 | 0.081 | Genotype |
| CX36 | 0.081 | Genotype |
| CASR | 0.093 | Allele |
| KCND1 | 0.094 | Allele |
| RARG | 0.095 | Genotype |
| PTGIR | 0.097 | Allele |
| CACNA2D3 | 0.103 | Genotype |
| CTSC | 0.104 | Genotype |
| ANXA5 | 0.111 | CHM |
| KCNQ1 | 0.112 | Allele |
| TRPV3 | 0.112 | Allele |
| LYN | 0.116 | CHM |
| CLN2 | 0.116 | Allele |
| PIM2 | 0.123 | Allele |
| KCNJ6 | 0.125 | Allele |
| IGSF11 | 0.132 | Genotype |
| SCN9A | 0.209 | Allele |
| TRPM3 | 0.209 | Genotype |
| PDE4D | 0.265 | Genotype |
| GRIN2B | 0.274 | Allele |
| ADAMTS16 | 0.304 | Genotype |
| HDAC4 | 0.527 | CHM |
| ESRRG | 0.618 | CHM |

TABLE 5
Detailed analysis results for the 21 genes significant following the permutation test.

| | | | Case | Control | Suspect | OR | | Primary | Primary OR |
|---|---|---|---|---|---|---|---|---|---|
| | NCBI | | Allele/ | Allele/ | Allele/ | (Lower Cl | Primary | Suspect | (Lower Cl |
| Gene-Poly | chr: position | P-value | Geno Freq | Geno Freq | Geno | Upper Cl) | P-value | Allele/Geno | Upper Cl) |
| BRAF | KINASE | v-raf murine sarcoma viral oncogene homolog B1 | | | | | | | |
| 3798964 | 07: | 0.004106 | A: 0.148 (332) | A: 0.116 | A | 1.3139 | 0.005762 | A | 1.5594 |
| | 139816111 | | G: 0.852 (1918) | (273) | | (1.11-1.56) | | | (1.16-2.10) |
| | | | | G: 0.884 | | | | | |
| | | | | (2073) | | | | | |
| ITGA9 | INTEGRIN | integrin, alpha 9 | | | | | | | |
| 5387374 | 03: | 0.002049 | C: 0.639 (1382) | C: 0.685 | T | 1.2330 | 0.0348 | T | 1.2702 |
| | 37338015 | | T: 0.361 (782) | (1630) | | (1.09-1.39) | | | (1.03-1.57) |
| | | | | T: 0.315 | | | | | |
| | | | | (748) | | | | | |
| 3345780 | 03: | 0.473580 | C: 0.648 (1439) | C: 0.659 | T | 1.0502 | 0.0045 | T | 1.3602 |
| | 37348848 | | T: 0.352 (781) | (1577) | | (0.93-1.19) | | | (1.11-1.67) |
| | | | | T: 0.341 | | | | | |
| | | | | (815) | | | | | |
| 3356289 | 03: | 0.640207 | G: 0.599 (1361) | G: 0.606 | T | 1.0315 | 0.0217 | T | 1.2835 |
| | 37399010 | | T: 0.401 (913) | (1493) | | (0.92-1.16) | | | (1.05-1.57) |
| | | | | T: 0.394 | | | | | |
| | | | | (971) | | | | | |
| CHRNA1 | ION_CHANNEL | cholinergic receptor, nicotinic, alpha polypeptide 1 (muscle) | | | | | | | |
| 5577886 | 02: | 0.003554 | A: 0.586 (1325) | A: 0.631 | G | 1.2082 | 0.6842 | G | 1.0478 |
| | 175591072 | | G: 0.414 (937) | (1519) | | (1.07-1.36) | | | (0.85-1.29) |
| | | | | G: 0.369 | | | | | |
| | | | | (889) | | | | | |
| 5577882 | 02: | 0.008779 | G: 0.357 (811) | G: 0.318 | G | 1.1911 | 0.4127 | G | 1.1002 |
| | 175598947 | | T: 0.643 (1459) | (784) | | (1.06-1.34) | | | (0.89-1.36) |
| | | | | T: 0.682 | | | | | |
| | | | | (1680) | | | | | |
| *CNR1 | 7TM | cannabinoid receptor 1 (brain) | | | | | | | |
| 3498129, | 6: 88808677, | 0.002778 | | | NOT | | 0.5168 | | |
| 3701919 | 6: 88834977 | | | | A_A | | | | |
| *ADAMTS7 | PROTEASE | a disintegrin-like and metalloprotease (reprolysin type) with thrombospondin type 1 motif, 7 | | | | | | | |
| 4351419, | 15: 76656233, | 0.002906 | | | NOT | | 0.1137 | A_C_C | |
| 5215276 | 15: 76658358 | | | | T_C | | | | |
| GABRB2 | ION_CHANNEL | gamma-aminobutyric acid (GABA) A receptor, beta 2 | | | | | | | |
| 3226723 | 05: | 0.495439 | C, C: 0.047 (54) | C, C: | C, C | 1.2838 | 0.0677 | G, G | 1.4253 |
| | 160676476 | | C, G: 0.316 | 0.037 | | (0.86-1.91) | | | (1.06-1.91) |
| | | | (361) | (46) | | | | | |
| | | | G, G: 0.637 | C, G: | | | | | |
| | | | (729) | 0.319 | | | | | |
| | | | | (396) | | | | | |
| | | | | G, G: | | | | | |
| | | | | 0.644 | | | | | |
| | | | | (798) | | | | | |
| 3239273 | 05: | 0.007573 | C, C: 0.517 | C, C: | C, C | 1.2977 | 0.2841 | T, T | 1.4418 |
| | 160678499 | | (586) | 0.452 | | (1.10-1.53) | | | (0.89-2.35) |
| | | | C, T: 0.397 (450) | (553) | | | | | |
| | | | T, T: 0.086 (97) | C, T: | | | | | |
| | | | | 0.458 | | | | | |
| | | | | (560) | | | | | |
| | | | | T, T: | | | | | |
| | | | | 0.090 | | | | | |
| | | | | (110) | | | | | |
| 3239268 | 05: | 0.052974 | C, C: 0.059 (67) | C, C: | T, T | 1.2245 | 0.1177 | !T, T | 1,3350 |
| | 160684022 | | C, T: 0.354 | 0.061 (74) | | (1.04-1.44) | | | (1.00-1.78) |
| | | | T, T: 0.587 | C, T: | | | | | |
| | | | (666) | 0.402 | | | | | |
| | | | (401) | (488) | | | | | |
| | | | | T, T: | | | | | |
| | | | | 0.537 | | | | | |
| | | | | (653) | | | | | |
| 3307225 | 05: | 0.003656 | A, A: 0.472 | A, A: | A, A | 1.3310 | 0.1984 | !A, A | 1.2655 |
| | 160690092 | | (537) | 0.402 | | (1.13-1.57) | | | (0.95-1.69) |
| | | | A, G: 0.425 | (496) | | | | | |
| | | | (484) | A, G: | | | | | |
| | | | G, G: 0.103 (117) | 0.479 | | | | | |
| | | | | (592) | | | | | |
| | | | | G, G: | | | | | |
| | | | | 0.119 | | | | | |
| | | | | (147) | | | | | |
| ESPL1 | PROTEASE | extra spindle poles like 1 (S. cerevisiae) | | | | | | | |
| 3484611 | 12: | 0.016201 | A: 0.615 (1400) | A: 0.651 | G | 1.1705 | 0.0201 | A | 1.3005 |
| | 53399073 | | G: 0.385 (878) | (1594) | | (1.040-1.32) | | | (1.05-1.61) |
| | | | | G: 0.349 | | | | | |
| | | | | (854) | | | | | |
| *CASP8 | PROTEASE | caspase 8, apoptosis-related cysteine protease | | | | | | | |
| 4108856, | 2: 202065738, | 0.006319 | | | G_G | | 0.0086 | A_T | |
| 3906565 | 2: 202117101 | | | | | | | | |
| MAP3K9 | KINASE | mitogen-activated protein kinase kinase kinase 9 | | | | | | | |
| 5523333 | 14: | 0.018535 | C: 0.124 (281) | C: 0.100 | C | 1.2665 | 0.2056 | T | 1.2730 |
| | 69223699 | | T: 0.876 (1989) | (246) | | (1.06-1.52) | | | (0.90-1.80) |
| | | | | T: 0.900 | | | | | |
| | | | | (2206) | | | | | |
| ADAMTS17 | PROTEASE | a disintegrin-like and metalloprotease (reprolysin type) with thrombospondin type 1 motif, 17 | | | | | | | |
| 5274016 | 15: | 0.007119 | C, C: 0.205 | C, C: | C, C | 1.3608 | 0.1899 | !G, G | 1.3376 |
| | 98251513 | | (233) | 0.159 | | (1.10-1.68) | | | (0.99-1.80) |
| | | | C, G: 0.455 | (196) | | | | | |
| | | | (516) | C, G: | | | | | |
| | | | G, G: 0.340 | 0.509 | | | | | |
| | | | (386) | (625) | | | | | |
| | | | | G, G: | | | | | |
| | | | | 0.332 | | | | | |
| | | | | (408) | | | | | |
| 4257470 | 15: | 0.979326 | A, A: 0.326 | A, A: | G, G | 1.0225 | 0.0292 | A, A | 1.5626 |
| | 98267006 | | (372) | 0.326 | | (0.83-1.26) | | | (1.13-2.17) |
| | | | A, G: 0.494 | (401) | | | | | |
| | | | (564) | A, G: | | | | | |
| | | | | 0.498 | | | | | |
| | | | | (612) | | | | | |
| | | | | G, G: | | | | | |
| | | | | 0.176 | | | | | |
| | | | | (217) | | | | | |
| | | | G, G: 0.180 | | | | | | |
| | | | (205) | | | | | | |
| *NOS2A | NONSYSTEMS | nitric oxide synthase 2A (inducible, hepatocytes) | | | | | | | |
| 5443014, | 17: 25952192, | 0.004805 | | | A_G_C_C | | 0.0310 | A_G_C_C | |
| 4629717, | 17: 25958968, | | | | | | | | |
| 5639664, | 17: 25979703, | | | | | | | | |
| 5639670 | 17: 25989245 | | | | | | | | |
| ROR1 | KINASE | receptor tyrosine kinase-like orphan receptor 1 | | | | | | | |
| 3386072 | 01: | 0.009401 | A, A: 0.048 (55) | A, A: | A, A | 1.9545 | 0.0038 | !A, C | 1.6191 |
| | 63822586 | | A, C: 0.333 | 0.025 (31) | | (1.25-3.05) | | | (1.20-2.19) |
| | | | (381) | A, C: | | | | | |
| | | | C, C: 0.619 | 0.324 | | | | | |
| | | | (707) | (401) | | | | | |
| | | | | C, C: | | | | | |
| | | | | 0.651 | | | | | |
| | | | | (806) | | | | | |
| 5816570 | 01: | 0.288406 | C, C: 0.374 | C, C: | !T, T | 1.1725 | 0.1469 | !C, C | 1.3386 |
| | 63976359 | | (427) | 0.351 | | (0.94-1.46) | | | (0.99-1.81) |
| | | | | (431) | | | | | |
| | | | C, T: 0.470 | C, T: | | | | | |
| | | | (536) | 0.471 | | | | | |
| | | | T, T: 0.156 | (579) | | | | | |
| | | | (178) | T, T: | | | | | |
| | | | | 0.178 | | | | | |
| | | | | (219) | | | | | |
| GABRG2 | ION_CHANNEL | gamma-aminobutyric acid (GABA) A receptor, gamma 2 | | | | | | | |
| 3232237 | 05: | 0.006772 | C: 0.599 (1336) | C: 0.641 | T | 1.1949 | 0.1438 | C | 1.1748 |
| | 161434280 | | T: 0.401 (896) | (1568) | | (1.06-1.34) | | | (0.96-1.44) |
| | | | | T: 0.359 | | | | | |
| | | | | (880) | | | | | |
| 3234461 | 05: | 0.567003 | C: 0.428 (970) | C: 0.437 | T | 1.0376 | 0.3752 | C | 1.1044 |
| | 161455422 | | T: 0.572 (1296) | (1064) | | (0.92-1.16) | | | (0.90-1.35) |
| | | | | T: 0.563 | | | | | |
| | | | | (1370) | | | | | |
| 3270990 | 05: | 0.182612 | C: 0.810 (1783) | C: 0.793 | C | 1.1128 | 0.0284 | T | 1.3414 |
| | 161497547 | | T: 0.190 (419) | (1904) | | (0.96-1.29) | | | (1.05-1.72) |
| | | | | T: 0.207 | | | | | |
| | | | | (498) | | | | | |
| 3257171 | 05: | 0.863844 | C: 0.645 (1442) | C: 0.642 | C | 1.0116 | 0.0272 | T | 1.2763 |
| | 161518204 | | T: 0.355 (794) | (1499) | | (0.90-1.14) | | | (1.04-1.57) |
| | | | | T: 0.358 | | | | | |
| | | | | (835) | | | | | |
| APP | NONSYSTEMS | amyloid beta (A4) precursor protein (protease nexin-II, Alzheimer disease) | | | | | | | |
| 3095740 | 21: | 0.005288 | A: 0.377 (854) | A: 0.420 | C | 1.1987 | 0.8801 | A | 1.0190 |
| | 26179530 | | C: 0.623 (1414) | (1018) | | (1.07-1.35) | | | (0.83-1.25) |
| | | | | C: 0.580 | | | | | |
| | | | | (1406) | | | | | |
| 3111653 | 21: | 0.584023 | C: 0.275 (618) | C: 0.267 | C | 1.0399 | 0.1098 | T | 1.2219 |
| | 26185463 | | T: 0.725 (1630) | (653) | | (0.91-1.18) | | | (0.97-1.54) |
| | | | | T: 0.733 | | | | | |
| | | | | (1791) | | | | | |
| 5154005 | 21: | 0.570275 | G: 0.129 (295) | G: 0.123 | G | 1.0566 | 0.1444 | G | 1.2964 |
| | 26190407 | | T: 0.871 (1999) | (301) | | (0.89-1.25) | | | (0.93-1.81) |
| | | | | T: 0.877 | | | | | |
| | | | | (2155) | | | | | |
| 3097557 | 21: | 0.011581 | C: 0.726 (1654) | C: 0.761 | T | 1.2004 | 0.0514 | T | 1.2728 |
| | 26250045 | | T: 0.274 (624) | (1887) | | (1.05-1.37) | | | (1.01-1.60) |
| | | | | T: 0.239 | | | | | |
| | | | | (593) | | | | | |
| 5155051 | 21: | 0.643516 | G: 0.315 (723) | G: 0.308 | G | 1.0333 | 0.4740 | T | 1.0864 |
| | 26333818 | | T: 0.685 (1573) | (766) | | (0.91-1.17) | | | (0.88-1.34) |
| | | | | T: 0.692 | | | | | |
| | | | | (1722) | | | | | |
| *PTGER3 | 7TM | prostaglandin E receptor 3 (subtype EP3) | | | | | | | |
| 3395306, | 1: 70791415, | 0.012299 | | | G_A | | 0.0946 | G_T_T_C_T | |
| 3330034 | 1: 70862039 | | | | | | | | |
| EDN1 | 7TM_LIGAND | endothelin 1 | | | | | | | |
| 3333322 | 06: | 0.111583 | A, A: 0.010 (11) | A, A: | !A, A | 2.0974 | 0.0444 | G, G | 1.5378 |
| | 12356804 | | A, G: 0.222 | 0.020 (25) | | (1.04-4.23) | | | (1.09-2.16) |
| | | | (255) | A, G: | | | | | |
| | | | | 0.216 | | | | | |
| | | | | (265) | | | | | |
| | | | G, G: 0.768 | G, G: | | | | | |
| | | | (881) | 0.763 | | | | | |
| | | | | (936) | | | | | |
| 3775084 | 06: | 0.015123 | C, C: 0.515 | C, C: | !T, T | 1.5656 | 0.2756 | C, C | 1.2657 |
| | 12357408 | | (586) | 0.507 | | (1.16-2.12) | | | (0.96-1.68) |
| | | | (478) | (620) | | | | | |
| | | | C, T: 0.420 | C, T: | | | | | |
| | | | T, T: 0.064 (73) | 0.396 | | | | | |
| | | | | (485) | | | | | |
| | | | | T, T: | | | | | |
| | | | | 0.097 | | | | | |
| | | | | (119) | | | | | |
| ITPR2 | ION_CHANNEL | inositol 1,4,5-triphosphate receptor, type 2 | | | | | | | |
| 3682469 | 12: | 0.452554 | C, C: 0.248 | C, C: | C, C | 1.1246 | 0.2500 | G, G | 1.2989 |
| | 26393180 | | (285) | 0.227 | | (0.93-1.36) | | | (0.95-1.77) |
| | | | C, G: 0.483 | (281) | | | | | |
| | | | (555) | C, G: | | | | | |
| | | | G, G: 0.268 | 0.505 | | | | | |
| | | | (308) | (625) | | | | | |
| | | | | G, G: | | | | | |
| | | | | 0.268 | | | | | |
| | | | | (332) | | | | | |
| 3559321 | 12: | 0.454307 | C, C: 0.114 | C, C: | T, T | 1.1080 | 0.1936 | !C, T | 1.2936 |
| | 26634912 | | (130) | 0.114 | | (0.94-1.30) | | | (0.97-1.72) |
| | | | C, T: 0.449 | (141) | | | | | |
| | | | (511) | C, T: | | | | | |
| | | | T, T: 0.436 | 0.474 | | | | | |
| | | | (496) | (585) | | | | | |
| | | | | T, | | | | | |
| | | | | T: 0.411 | | | | | |
| | | | | (507) | | | | | |
| 3703807 | 12: | 0.389704 | A, A: 0.354 | A, A: | G, G | 1.1713 | 0.1291 | A, A | 1.3039 |
| | 26701503 | | (402) | 0.359 | | (0.94-1.46) | | | (0.97-1.75) |
| | | | A, G: 0.478 | (439) | | | | | |
| | | | | A, G: | | | | | |
| | | | (543) | 0.494 | | | | | |
| | | | G, G: 0.167 | (604) | | | | | |
| | | | | G, G: | | | | | |
| | | | | 0.146 | | | | | |
| | | | | (179) | | | | | |
| | | | (190) | | | | | | |
| 4896738 | 12: | 0.045844 | C, C: 0.915 | C, C: | !T, T | 5.2933 | | C, T | 1.3042 |
| | 26707221 | | (1051) C, T: | 0.896 | | (0.93-30.09) | | | (0.84-2.03) |
| | | | 0.084 (97) T, T: | (1108) | | | | | |
| | | | 0.001 (1) | C, T: | | | | | |
| | | | C, T: 0.084 (97) | 0.098 | | | | | |
| | | | T, T: 0.001 (1) | (121) | | | | | |
| | | | | T, T: | | | | | |
| | | | | 0.006 (8) | | | | | |
| 4896735 | 12: | 0.226477 | C, C: 0.549 | C, C: | !C, C | 1.1550 | 0.0763 | C, C | 1.3832 |
| | 26707222 | | (619) | 0.584 | | (0.98-1.36) | | | (1.04-1.84) |
| | | | C, T: 0.391 | (715) | | | | | |
| | | | (441) | C, T: | | | | | |
| | | | T, T: 0.060 (68) | 0.364 | | | | | |
| | | | | (445) | | | | | |
| | | | | T, T: | | | | | |
| | | | | 0.052 (64) | | | | | |
| 3549644 | 12: | 0.204583 | C, C: 0.680 | C, C: | C, C | 1.1720 | 0.0337 | !C, C | 1.4829 |
| | 26710494 | | (773) | 0.645 | | (0.99-1.39) | | | (1.10-2.01) |
| | | | C, T: 0.285 | (792) | | | | | |
| | | | (324) | C, T: | | | | | |
| | | | T, T: 0.034 | 0.315 | | | | | |
| | | | (39) | (387) | | | | | |
| | | | | T, T: | | | | | |
| | | | | 0.040 (49) | | | | | |
| 3686945 | 12: | 0.596742 | C, C: 0.710 | C, C: | C, C | 1.0974 | 0.5368 | !C, C | 1.2220 |
| | 26720004 | | (810) | 0.690 | | (0.92-1.31) | | | (0.87-1.72) |
| | | | C, T: 0.267 | (845) | | | | | |
| | | | (305) | C, T: | | | | | |
| | | | T, T: 0.023 | 0.286 | | | | | |
| | | | (26) | (350) | | | | | |
| | | | | T, T: | | | | | |
| | | | | 0.024 (29) | | | | | |
| 3433876 | 12: | 0.135071 | A, A: 0.125 | A, A: | C, C | 1.1863 | 0.0354 | !C, C | 1.4664 |
| | 26722619 | | (142) | 0.133 | | (1.01-1.40) | | | (1.10-1.96) |
| | | | A, C: 0.429 | (164) | | | | | |
| | | | (487) | A, C: | | | | | |
| | | | C, C: 0.446 | 0.463 | | | | | |
| | | | (507) | (572) | | | | | |
| | | | | C, C: | | | | | |
| | | | | 0.405 | | | | | |
| | | | | (500) | | | | | |
| 3640150 | 12: | 0.787044 | C, C: 0.227 | C, C: | !C, C | 1.0687 | 0.7804 | T, T | 1.1283 |
| | 26768589 | | (261) | 0.238 | | (0.88-1.29) | | | (0.81-1.57) |
| | | | (573) | (294) | | | | | |
| | | | C, T: 0.497 | C, T: | | | | | |
| | | | T, T: 0.276 | 0.487 | | | | | |
| | | | (318) | (600) | | | | | |
| | | | | T, T: | | | | | |
| | | | | 0.275 | | | | | |
| | | | | (339) | | | | | |
| 3670055 | 12: | 0.002518 | A, A: 0.254 | A, A: | G, G | 1.4146 | 0.1761 | A, G | 1.2971 |
| | 26847121 | | (288) | 0.282 | | (1.17-1.71) | | | (0.97-1.73) |
| | | | A, G: 0.484 | (345) | | | | | |
| | | | (550) | A, G: | | | | | |
| | | | G, G: 0.262 | 0.518 | | | | | |
| | | | (298) | (634) | | | | | |
| | | | | G, G: | | | | | |
| | | | | 0.201 | | | | | |
| | | | | (246) | | | | | |
| ACCN1 | ION_CHANNEL | amiloride-sensitive cation channel 1, neuronal (degenerin) | | | | | | | |
| 4261588 | 17: | 0.662929 | A: 0.415 (947) | A: 0.422 | T | 1.0283 | 0.8818 | A | 1.0175 |
| | 31371652 | | T: 0.585 (1333) | (1041) | | (0.92-1.15) | | | (0.83-1.25) |
| | | | | T: 0.578 | | | | | |
| | | | | (1425) | | | | | |
| 4301609 | 17: | 0.014416 | A: 0.907 (2087) | A: 0.884 | A | 1.2896 | 0.3992 | T | 1.1557 |
| | 32286024 | | T: 0.093 (213) | (2202) | | (1.07-1.55) | | | (0.85-1.58) |
| | | | | T: 0.116 | | | | | |
| | | | | (290) | | | | | |
| USP9X | PROTEASE | ubiquitin specific protease 9, X-linked (fat facets-lile, Drosophila) | | | | | | | |
| 3148022 | X: | 0.03230 | A, A: 0.008 (4) | A, A | A, A | 10.7297 | 0.2618 | G, G | 2.3601 |
| | 39839143 | | A, G: 0.137 (69) | 0.000 | | (0.58-199.77) | | | (1.07-5.18) |
| | | | G, G: 0.855 | (0) | | | | | |
| | | | (430) | A, G: | | | | | |
| | | | | 0.170 | | | | | |
| | | | | (101) | | | | | |
| | | | | G, G: | | | | | |
| | | | | 0.830 | | | | | |
| | | | | (494) | | | | | |
| VIP | 7TM_LIGAND | vasoactive intestinal peptide | | | | | | | |
| 3647066 | 06: | 0.017250 | A, A: 0.041 (44) | A, A: | !A, T | 1.3604 | 0.1588 | !T, T | 1.3510 |
| | 153020455 | | A, T: 0.290 | 0.036 | | (1.11-1.67) | | | (1.00-1.82) |
| | | | (312) | (24) | | | | | |
| | | | T, T: 0.669 | A, T: | | | | | |
| | | | (720) | 0.357 | | | | | |
| | | | | (240) | | | | | |
| | | | | T, T: | | | | | |
| | | | | 0.607 | | | | | |
| | | | | (408) | | | | | |
| 4577701 | 06: | 0.613650 | A, A: 0.110 | A, A: | G, G | 1.0883 | 0.0465 | A, A | 1.7277 |
| | 153037500 | | (124) | 0.114 | | (0.92-1.28) | | | (1.10-2.71) |
| | | | A, G: 0.423 | (136) | | | | | |
| | | | (479) | A, G: | | | | | |
| | | | G, G: 0.467 | 0.440 | | | | | |
| | | | (529) | (524) | | | | | |
| | | | | G, G: | | | | | |
| | | | | 0.446 | | | | | |
| | | | | (532) | | | | | |
| ADAM19 | PROTEASE | a disintegrin and metalloproteinase domain 19 (meltrin beta) | | | | | | | |
| 6210770 | 05: | 0.039985 | C: 0.735 (1671) | C: 0.763 | G | 1.1623 | 0.2584 | G | 1.1521 |
| | 156866327 | | G: 0.265 (603) | (1865) | | (1.02-1.33) | | | (0.91-1.45) |
| | | | | G: 0.237 | | | | | |
| | | | | (579) | | | | | |

### REFERENCES

Adam M. et al. (1994), Cloning and expression of three isoforms of the human EP3 prostanoid receptor. FEBS Letters 338: 170-174. 1994
Balon T W, and Nadler J L. 1994. Nitric oxide release is present from incubated skeletal muscle preparations. J. Appl. Physiol. 77: 2519-2521. 1994
Balon T W, and Nadler J L. 1997. Evidence that nitric oxide increases glucose transport in skeletal muscle. J. Appl. Physiol. 82: 359-363. 1997
Baron A D (2002), Insulin resistance and vascular function. Journal of Diabetes and it's Complications, 16:92-102.2002
Bedard S., Marcotte B. and Marett A. 1997. Cytokines modulate glucose transport in skeletal muscle by inducing the expression of inducible nitric oxide synthase. Biochem. J. 325: 487-493. 1997
Brock B (1999), The insulinotropic effect of endothelin-1 is mediated by glucagon release from the islet alpha cells. Diabetoligia 42: 1302-1307. 1999
Carey A L, et al (2004), Interleukin-6 and tumor necrosis factor-□ are not increased in patients with type 2 diabetes. Diabetologia, 47: 1029-1037. 2004
Chadee, D. N., and Kyriakis, J. M. 2004. MLK3 is required for mitogen activation of B-RAF, ERK, and cell proliferation. Nature Cell Biol. 6: 770-776. 2004
Chebib M, and Johnston G. 1999. The “ABC” of GABA receptors: A brief review. 1999. Clinical and experimental Pharmacology and Physiology: 26: 937-940. 1999
Chiang S-H, et al (2003), TCGAP, a multidomain rho GTPase-activating protein involved in insulin-stimulated glucose transport. EMBO Journal, 22: 2679-2691. 2003
Clarke K J, et al (2003), Regulation of adiponectin secretion by endothelin-1. Biochemical Biophysical Research Communications 312: 945-949. 2003
Corbett J A, et al 1993. Nitric oxide mediates cytokine-induced inhibition of insulin secretion by human islets of langerhans. Proc. Natl. Acad. Sci. USA 90:1731-1735. 1993
Cottet S, et al. 2002. cFLIP protein prevents tumor necrosis factor-alpha-mediated induction of caspase-8-dependent apoptosis in insulin-secreting betaTc-Tet cells. Diabetes. 51:1805-1814. 2002
DeAizpurua H J, et al 1997. Expression of mixed lineage kinase-1 in pancreatic □-cell lines at different stages of maturation and during embryonic pancreas development. J. Biol. Chem. 272: 16364-16373. 1997
Delgado M, et al (1999), vasoactive intestinal peptide and ituitary adenylate cyclase-activating polypeptide inhibit endotoxin-induced TNF-□ production by macrophages. Journal of Immunology, 162: 2358-2367. 1999
Delgado M, Gavera D (2003), Vasoactive intestinal peptide inhibites IL-8 production in human monocytes. Biochemistry and Biophysics Research Communications, 301: 825-832. 2003
De Mora J F, et al 2003. Ras-GRF1 signalling is required for normal □-cell development and glucose homeostasis. EMBO J. 22: 3039-3049. 2003
Devlin B. Roeder K. Wasserman L (2002). Genomic control, a new approach to genetic-based association studies Theoretical Population Biology. 60:155-66, 2001.
Duncan B B, et al. (2003), Low-grade systemic inflammation and the development of type 2 diabetes. Diabetes 52: 1799-1805. 2003
Ehses J A, Pelech S L, Pederson R A, and McIntosh CHS. 2002. Glucose-dependent insulinotropic polypeptide activates the Raf-Mek1/2-ERK1/2 module via a cyclic AMP/cAMP-dependent protein kinase/Rap1-mediated pathway. J. Biol. Chem. 40: 37088-37097. 2002
Fahrenkrug J, Emson P C (1982), Vasoactive intestinal peotide: functional aspects. British Medical Bulletin, 38: 265-270. 1982
Figueroa D J, Shi X-P, Gardell S J, and Austin C P. 2001. A□pp secretases are co-expressed with A□pp in the pancreatic islets. J. Alzheimers Disease 3: 393-396. 2001
Fujimoto M, Martyn J A J, Kaneki M. 2004. iNOS inhibitor, L-Nil ameliorated hyperglycemia and insulin resistance, and improved hepatic insulin signaling in obese (Ob/Ob) mice. American Diabetes Association Abstract ADA4L_{—}1512B. 2004
Gallo, K. A. and Johnson, G. L. 2002. Mixed-lineage kinase control of JNK and p38 MAPK pathways. Mol. Cell. Biol. 3: 663-672. 2002
Gregersen S, Thomsen J L, Hermansen K (2000), Endothelin-1 (ET-1)-potentiated insulin secretion: involvement of protein kinase C and the ETA receptor subtype. Metabolism 49.264-269. 2000
Harris, C. A. et al. 2001. Inhibition of c-Jun N-terminal kinase signaling pathway by the Mixed Lineage Kinase Inhibitor CEP-1347 (KT7515) preserves metabolism and growth of trophic factor-deprived neurons. Neuroscience 22: 103-113. 200
Hasewa H, Negishi M, Katoh H, Ichikawa A (1997), Two isoforms of prostaglandin EP3 receptor exhibiting constitutive activity and agonist-dependent activity in rho-mediated stress fiber formation. Biochem Biophys Res Comm 234: 631-636. 1997
Hauner H, Pertuschke T, Gries F A (1994), Endothelin-1 inhibits the adipose differentiation of cultured human adipocyte precursor cells. Metabolism 43: 227-232. 1994
Hirosumi J., et al. 2002. A central role for JNK in obesity and insulin resistance. Nature 420: 333-336. 2002
Imamura T, et al (1999), Endothelin-1-induced GLUT4 translocation is mediated via G□_{q/11} protein and phosphatidylinositol 3-kinase in 3T3-L1 adipocytes. Journal of Biological Chemistry, 274:33691-33695. 1999
Inomata H, et al 2003. A scaffold protein JIP-1b enhances amyloid precursor protein phosphorylation by JNK and its association with kinesin light chain. J. Biol. Chem. 278: 22946-22955. 2003
Inoue A, et al (1989), The human endothelin family: three structurally and pharmacologically distinct isopeptides predicted by three separate genes. Proceedings of the National Academy of Sciences (USA) 86: 2863-2867. 1989
Ishibashi K-I, et al. (2001), Chronic endothelin-1 treatment leads to heterologous desensitization of insulin signalin in 3T3-L1 adipocytes. Journal of Clinical Investigation 107:1193-1202. 2001
Itoh N, et al (1983), Human preprovasoactive intestinal polypeptide contains a novel PHI-27-like peptide, PHM-27. Nature 304: 547-549. 1983
Janson J, Laedtke et al. 2004. Increased risk of type II diabetes in Alzheimer disease. Diabetes 53: 474-481. 2004
Johnson K H, et al 1992. Islet amyloid polypeptide: mechanisms of amyloidogenesis in the pancreatic islets and potential roles in diabetes mellitus. Laboratory Investigation. 66(5):522-35, 1992.
Juan C-C, et al (1996), Endothelin-1 induces insulin resistance in conscious rats. Biochemical and Biophysical Research Communications 227: 694-699. 1996
Juan C-C, et al (1998), Overexpression of vascular endothelin-1 and endothelin-A receptors in a fructose-induced hypertensive rat model. Journal of Hypertension 16: 1775-1782. 1998
Kahn. Diabetologia. 2003; 46(1):3-19. 2003
Kang S, et al. 2003. Insulin can block apoptosis by decreasing oxidative stress via phosphatidylinositol 3-kinase- and extracellular signal-regulated protein kinase-dependent signaling pathways in HepG2 cells. Eur. J. of Endocrinol. 148:147 2003
Kapur S, et al 1997. Expression of nitric oxide synthase in skeletal muscle: a novel role for nitric oxide as a modulator of insulin action. Diabetes 46:1691. 1997
Kawarabayashi T, et al 1996. Accumulation of b-amyloid fibrils in pancreas of transgenic mice. Neurobiol. of Aging 17: 215-222.1996
Kolch W. 2001. To be or not to be: a question of B-Raf?. Trends in Neurosciences 24:498-500. 2001
Kotani M, et al (1995), Molecular cloning and expression of multiple isoforms of human prostaglandin E receptor EP3 subtype generated by alternative messenger RNA splicing Molecular Pharmacology, 48: 869-879. 1995
Kotani, M.; et al (1997), Structural organization of the human prostaglandin EP(3) receptor subtype gene (PTGER3). Genomics 40: 425-434.1997
Kotani M, et al., (2000), Multiple signal transduction pathways through two prostaglandin E receptor EP3 subtype isoforms expressed in human uterus. Journal of Clinical Endocrinology and Metabolism, 85: 4315-4322. 2000
Kuhn A (1971) Lectures on Developmental Physiology. New York: Springer-Verlag. 1971
Kurihara Y, Endo H, Kondo H (2001), Induction of IL-6 via the EP3 subtype of prostaglandin E receptor in rat adjuvant-arthritic synovial cells. Inflammation Research, 50:1-5. 2001
Kuroda M, et al. (1987), Regulation of insulin-stimulated glucose transport in the isolated rat adipocyte. Journal of Biological Chemistry, 262: 245-253. 1987
Lee Y-C, Juan et al., (1998), Evidence that endothelin-1 (ET-1) inhibits insulin-stimulated glucose uptake in rat adipocytes mainly through ET_{A} receptors. Metabolism 47:1468-1471.1998
Liebson C L, et al. 1997. The risk of dementia among persons with diabetes mellitus; a population based cohort study. Am. J. Epidemiol 145: 301-308. 1997
Lillioja S, et al., N Engl J. Med. 329:1988 (1993).
Liu J. Dragunow M. Cooper G J. 2003. Fibrillogenic amylin evokes islet beta-cell apoptosis through linked activation of a caspase cascade and JNK1. Journal of Biological Chemistry. 278:52810-52819.2003
Maedler K. et al 2001. Glucose induces beta-cell apoptosis via upregulation of the Fas receptor in human islets. Diabetes. 50:1683-1690. 2001
Maedler K et al. 2002. FLIP switches Fas-mediated glucose signaling in human pancreatic beta cells from apoptosis to cell replication. Proceedings of the National Academy of Sciences of the United States of America. 99: 8236-8241. 2002
Mandrup-Poulsen T. 2003. Apoptotic signal transduction pathways in diabetes. Biochem. Pharmacol. 66(8):1433-1440. 2003
Mater M K, Thelen A P, Jump D B (1999), Arachidonic acid and PGE2 regulation of hepatic lipogenic gene expression. J. Lipid Res., 40: 1045-1052. 1999
Mather K J, et al. (2002), Endothelin contributes to basal vascular tone and endothelial dysfunction in human obesity and type 2 diabetes. Diabetes, 51: 3517-3523. 2002
Mehta, C. and Patel, N. (1983) A network algorithm for performing Fisher's Exact Test in r×c contingency tables. J. Am. Stat. Assoc. 78, 427-434
Meng Z, et al. Selection of genetic markers for association analyses, using linkage disequilibrium and haplotypes. Am J Hum Genet. 71 (1) 115-130 (2003).
Mielke, K. and Herdegen, T. 2002. Fatal shift of signal transduction is an integral part of neuronal differentiation. Mol. and Cell. Neuroscience 20: 211-224. 2002
Mootha V K, et al. (2003), PGC1a-responsive genes involved in oxidative phosphorylation are coordinately downregulated in human diabetes. Nature Genetics, 34: 267-273. 2003
Nadler S T, et al (2000), The expression of adipogenic genes is decreased in obesity and diabetes mellitus. Proceedings of the National Academy of Sciences (USA), 97(21) 11371-11376. 2000
Namba T, et al. (1993), Alternative splicing of C-terminal tail of prostaglandin E receptor subtype EP3 determines G-protein specificity. Nature, 365:166-170. 1993
Nicolls M R, et al. 2002. Proteomic as a tool for discovery: Proteins implicated in alzheimer's disease are highly expressed in normal pancreatic islets. J. Proteome Res.: 199-205. 2002
Ottosson-Seeberger A, et al (1997), Exogenous endothelin-1 causes peripheral insulin resistance in healthy humans. Acta Physiologia Scandinavia 161: 211-220. 1997
Park H S, and Park H J. 2000. Effects of g-aminobutyric acid on secretagogue-induced exocrine secretion of isolated, perfused rat pancreas. Am. J. Phsiolo. Gastrointest Liver Physiol. 279: G677-G682. 2000
Patti M E, et al. (2003), Coordinated reduction of genes of oxidative metabolism in humans with insulin resistance and diabetes: potential role of PGC1 and NRF1. PNAS (USA), 100 (14) 8466-8471. 2003
Paviovic D, et al 2000, Activation of extracellular signal-regulated kinase (ERK) ½ contributes to cytokine-induced apoptosis in purified rat pancreatic beta-cells. European Cytokine Network. 11:267-274. 2000
Perreault M, and Marett A. 2001. Targeted disruption of inducible nitric oxide synthase protects against obesity-linked insulin resistance in muscle. Nature Medicine 7: 1138-1143. 2001
Pierucci D, et al 2001. NGF-withdrawal induces apoptosis in pancreatic beta cells in vitro. Diabetologia 44: 1281-1295. 2001
Puschel G P, et al (1993), Glycogenolytic and antiglycogenolytic prostaglandin E2 actions in rat hepatocytes are mediated via different signalling pathways. European Journal of Biochemistry, 218: 1083-1089.1993
Rachdi L, Marie J-C, Scharfmann R (2003), Role for VPAC2 receptor-mediated signals in pancreas development. Diabetes 52: 85-92. 2003
Richelsen B, Eriksen E F, Beck-Nielsen H, Pedersen 0 (1984), Prostaglandin E2 receptor binding and action in human fat cells. Journal of Clinical Endocrinology and Metabolism, 59: 7-12. 1984
Richelsen B, Beck-Nielsen H (1984), Decrease of prostaglandin E2 receptor biding is accompanied by reduced antilipolytic effects of prostaglandin E2 in isolated rat adipocytes. J Lipid Research 26: 127-134. 1984
Richelsen B, et al (1985), Effects of prostaglandin E2, indomethacin and adenosine deaminase on basal and insulin-stimulated glucose metabolism in human adipocytes. Biochim Biophys Acta 844: 359-366. 1985
Rotter V, Nagaev I, Smith U (2003), Interleukin-6 (IL-6) induces insulin resistance in 3T3-L1 adipocytes and is, like IL-8 and tunor necrosis factor-□, overexpressed in human fat cells from insulin-resistant subjects. Journal of Biological Chemistry, 278: 45777-45784. 2003
Sakurai T, et al. (1990), Cloning of a cDNA encoding a non-isopeptide-selective subtype of the endothelin receptor. Nature 348: 732-735. 1990
Schmid A, et al. (1995), Splice variants of the human EP3 receptor for prostaglandin E2. European Journal of Biochemistry, 228: 24-30. 1995
Senn J J, et al (2002), Interieukin-6 induces cellular insulin resistance in hepatocytes. Diabetes, 51: 3391-3399. 2002
Shang J, et al (2004) Distinct patterns of transcriptional regulation in skeletal muscle and adipose tissue form humans with type 2 diabetes: evidence of a role of inflammation. Diabetes 53 (suppl 2) A266. 2004
Shimabukuro M, et al. 1997. Role of nitric oxide in obesity-induced □-cell disease. J. Clin. Invest. 100: 290-295. 1997
Shum W W C, Le G-y, Jones R L, Gurney A M, Sasaki Y (2003), Involvement of rho-kinase in contraction of guinea-pig aorta induced by prostanoid EP3 receptor agonists. British Journal of Pharmacology, 139: 1449-1461. 2003
Steinberg H O, et al (1996), Obesity/insulin resistance is associated with endothelial dysfunction: implications for the syndrome of insulin resistance. Journal of Clinical Investigation, 97 (11) 2601-2610. 1996
Taylor et al, Flow Cytometric platform for high-throughput single nucleotide polymorphism analysis. BioTechniques 30 (3) 661-669 (2001)
Thomson F J, et al (1997), Characterization of the intracellular signalling pathways that underlie growth-factor-stimulated glucose transport in Xenopus oocytes: evidence for ras- and rho-dependent pathways of phosphatidylinositol 3-kinase activation. Biochemical Journal 325: 637-643. 1997
Todaro M, et al 2003. Islet beta-cell apoptosis triggered in vivo by interleukin-1 beta is not related to the inducible nitric oxide synthase pathway: evidence for mitochondrial function impairment and lipoperoxidation. Endocrinology. 144:4264-4271. 2003
Tran P O T, et al (1999), Prostaglandin E2 mediates inhibition of insulin scretion by interleukin-1□. Journal of Biological Chemistry, 274: 31245-31248. 1999
Tran P O T, Gleason C E, Robertson R P (2002), Inhibition of interleukin-1□-induced COX-2 and EP3 gene expression by sodium salicylate enhances pancreatic islet D-cell function. Diabetes, 51: 1772-1778. 2002
Trinkaus J P (1969) Cells into Organs: Forces that Shape the Embryo. Englewood Cliffs, N.J.: Prentice-Hall, Inc. 1969
Uriguen L, et al 2004. Impaired action of anxiolytic drugs in mice deficient in cannabinoid CB₁ receptors. Neuropharmacol. 46: 966-973. 2004
van den Berghe N, et al. (1996), Clostridium botulinum C3 exoenzyme stimulates GLUT4-mediated glucose transport, but not glycogen synthesis, in 3T3-L1 adipocytes—a potential role of rho? Biochem Biophys Res Comm 229: 430-439.1996
Verchere C B. Et al. 1996. Islet amyloid formation associated with hyperglycemia in transgenic mice with pancreatic beta cell expression of human islet amyloid polypeptide. PNAS (USA) 93(8):3492-3496. 1996
Watson S, Arkinstall S. Vasoactive Intestinal Polypeptide Family: The G-protein Linked Receptor Factsbook. London, Academic Press, 1994
Wei Y, Mojsov S (1996), Tissue specific expression of different human receptor types for pituitary adenylate cyclase activating polypeptide and vasoactive intestinal polypeptide: implications for their role in human physiology. Journal of Neuroendocrinology, 8: 811-817. 1996
Weisberg S P, et al 2003. Obesity is associated with macrophage accumulation in adipose tissue. J. Clin. Invest. 112:1796-1808. 2003
Wendt A. et al. 2004. Glucose inhibition of glucagon secretion from rat □-cells is mediated by GABA released from neighboring □-cells. Diabetes 53: 1038. 2004
Williams N G and Roberts T M. 1994. Signal transduction pathways involving the Raf proto-oncogene. Cancer & Metastasis Reviews. 13:105-116 1994
Wilson J D, Foster D W, Williams Textbook of Endocrinology. Philadelphia, Pa. WB Saunders Company, 1992
Wojnowski L, et al. 1997. Endothelial apoptosis in Braf-deficient mice. Nature Genetics. 16:293-297. 1997
Wu-Wong J, et al (1999), Endothelin stimulates glucose uptake via activation of endothelin-A receptor in 3T3-L1 adipocytes. Journal of Biological Chemistry, 274: 8103-8110. 1999
Xu, A. et al. 2001. The MLK family mediated c-Jun terminal kinase activation in neuronal apoptosis. Molec. Cell. Biol. 21: 4713-4724. 2001
Yamaguchi T, Kubota T, Watanabe S, Yamamoto T (2004), Activation of brain prostanoid EP3 receptors via arachidonic acid cascade during behavioral suppression induced by d8-tetrahydrocannabinol. J. Neurochem. 88:148-154. 2004
Yamaguchi T, et al., Nature Medicine 7: 941-946, 2001
Yanagisawa M, et al (1988), A novel potent vasoconstrictor peptide produced by cells. Nature 332: 411-415. 1988
Yang W, Reyes A A, and Lan, N C. 1994. Identification of the GABA_{A} receptor subtype mRNA in human pancreatic tissue. FEBS Letter 346: 257-262. 1994
Zaykin D, Ehm M, Weir, B (2005). The composite haplotype method for association mapping of complex traits in outbred populations. (In press)
Zaykin D, Zhivotovsky L, Weir B S. Exact tests for association between alleles at arbitrary numbers of loci. Genetica 96:169-78. 1995

That which is claimed is:

1. A method of screening small molecule compounds for use in treating Type II Diabetes Mellitus (T2DM), comprising screening a test compound against a target selected from the group consisting of Prostaglandin E Receptor 3 (EP3), inducible Nitric Oxide Synthase (iNOS) BRAF, Caspase 8, Integrin Alpha 9 (ITGA9), ADAMTS7 (A Disintegrin-like And Metalloprotease domain with Thrombospondin motifs 7) and Amyloid Precursor Protein, where activity against said target indicates the test compound has potential use in treating Type II Diabetes Mellitus.

2. A method according to claim 1 wherein the target is EP3.

3. A method of lowering serum blood glucose levels in a mammalian subject in need thereof, comprising administering to said subject an EP3 antagonist in an amount effective to decrease serum blood glucose levels compared to the level of serum blood glucose that would be seen in the absence of said administration.

4. A method according to claim 3 wherein the subject is a human with Type II diabetes mellitus (T2D).

5. A method of increasing glucose uptake from the blood in a mammalian subject in need thereof, comprising administering to said subject an EP3 antagonist in an amount effective to increase uptake of blood glucose compared to the level of uptake that would be seen in the absence of said administration.

6. A method according to claim 5 wherein the subject is a human with Type II diabetes mellitus (T2D).
